(19) United States
(12) Patent Application Publication
(10) Pub. No.: US 2022/0008511 A1
(43) Pub. Date: Jan. 13, 2022

(54) TUMOR TREATMENT USING CYTOKINES AND CANCER DRUGS

(71) Applicant: Henry J. Smith, Temecula, CA (US)

(72) Inventor: Henry J. Smith, Temecula, CA (US)

(21) Appl. No.: 16/926,767

(22) Filed: Jul. 12, 2020

(51) Int. Cl. A61K 38/19; A61K 9/00; A61K 47/69; A61K 38/17; A61K 38/18; A61K 38/40; C07K 16/22; C07K 16/28

(52) U.S. Cl. CPC A61K 38/191; A61K 9/0019; A61K 47/6911; C07K 16/2863; A61K 38/1866; A61K 38/40; C07K 16/22; A61K 38/179

(57) ABSTRACT

This invention discloses a pharmaceutical composition for treating tumors wherein said pharmaceutical comprises a proinflammatory cytokine such as Tumor Necrosis Factor alpha (TNF-a) combined with one or more small molecule cancer drugs within the same liposome. The liposomes are sized to be below 250 nm in diameter to enable them to localize within the tumor due to the Enhanced Permeability and Retention (EPR) effect. This liposomal formulation will ensure that the proinflammatory cytokine and the cancer drug are localized together within the tumor and with less exposure to normal tissues. This invention also discloses that the safety and efficacy of said proinflammatory cytokine/drug liposomes could be further enhanced by coating the exterior of said liposomes with a tumor targeting agent.

### CROSS REFERENCE TO RELATED APPLICATIONS

This patent application claims priority to Provisional Patent Application No. 62/922,570 titled “Combination Therapy using Tumor Necrosis Factor and Cancer Drugs” and filed Aug. 16, 2019.

### STATEMENT RE: FEDERALLY SPONSORED RESEARCH/DEVELOPMENT

Not Applicable

### BACKGROUND INFORMATION

Tumor Necrosis Factor alpha (TNF-a) is a cytokine with multiple effects. It is involved in systemic inflammation and is one of the cytokines involved in the acute phase reaction. Early studies showed that administration of TNF-a to animals bearing tumors resulted in extensive necrosis of the tumor (Carswell et al. 1975; Creasey et al. 1986). Subsequent testing of TNF-a as a cancer drug however proved disappointing. In certain studies administration of TNF-a appeared to have little or no affect upon the tumor and in some cases it even appeared to stimulate tumor growth and metastasis.

Increasing the amount of TNF-a in order to obtain a more cytotoxic effect upon the tumor was not successful as increased levels of TNF-a was often associated with inducing shock-like symptoms such as fever, chills and pain (Selby et al. 1987; Creagan et al. 1988; Brown et al. 1991; Furman et al. 1993).

However in certain situations where the tumor was located on a limb that could be isolated perfusing the limb with a high dose of TNF-a would sometimes result in tumor inhibition, especially if the patient was being treated with a cancer drug at the same time (Eggermont et al. 1996; de Wilt et al. 1999; Lienard et al. 1992; Lejeune et al 1995; Fraker et al 1996). It was also noted that even in those cases where the tumor is located in a site that cannot be isolated and perfused, even limited doses of TNF-a appeared to potentiate the cytotoxic effect of a cancer drug upon tumor growth (Curnis et al. 2002).

Cancer patients undergoing chemotherapy are typically administered one or more small molecule cancer drugs. Although these drugs are effective against the cancer they are frequently accompanied by severe side-effects to the patient. This is because the cancer drugs are able to penetrate into the tumor and also into normal tissues and harm normal cells. Enclosing the cancer drug within a liposome that was sized between 100 nm and 400 nm and administering the liposomal drug intravenously prevented the drug from entering normal tissues and causing harm. This was because the drug incorporated liposomes are too large to extravasate through the endothelial pores of normal blood vessels. However the drug incorporated liposomes are small enough to exit through the enlarged endothelial pores of the leaky blood vessels supplying the tumor and localize within the tumor where the cancer drug is released. This is known as the “Enhanced Permeability and Retention” (EPR) effect (Maruyama 2011). Incorporating a cancer drug within a liposome often leads to a significant improvement in safety and efficacy.

TNF-a is a water-soluble compound that can enter into normal tissues and cause harm especially when used in large doses. This invention teaches that combining TNF-a with a cancer drug within the same liposome would thereby prevent the TNF-a from also entering normal tissues and causing harmful side-effects. It would also prolong the bioavailability of the TNF-a, and also an increased localization of the TNF-a within the tumor because of the EPR effect.

The novelty of this invention is that it teaches a method of treating tumors using a pharmaceutical formulation that incorporates a combination of TNF-a and one or more small molecule cancer drugs within a liposome. The delivery of the combined TNF-a and the cancer drug together to the tumor will have a synergistic cytotoxic effect upon the tumor. At the same time enclosing both the TNF-a and the cancer drug within the liposome will also mitigate harmful side-effects to normal tissues. The liposomes are sized to be between 100-250 nm in diameter and preferably to be about 100-150 nm in order take advantage of the EPR effect and localize within the tumor. In addition to the direct cytotoxic effect of the TNF-a combined with the cancer drug has upon the tumor there is also a subsequent follow-on inflammatory response to the TNF-a within the tumor, and this may also contribute to inhibition of tumor growth. This increased cytotoxicity to the tumor is accompanied by an improvement in safety because the TNF-a/drug liposomes are unable to extravasate out of normal blood vessels and cause harm to normal tissues.

This invention further teaches that there are other proinflammatory cytokines such as Interleukin-1 beta (IL-1b), Interleukin-6 (IL-6), Interleukin-12 (IL-12) and Interleukin-18 (IL-18) that could similarly be combined with one or more cancer drugs within a liposome and used to treat a tumor.

This invention also teaches that the safety and efficacy of the proinflammatory cytokine/drug liposomes could be further improved by coating the exterior of said liposomes with a tumor targeting agent such as an anti-tumor antibody, or an anti-tumor aptamer, or an anti-tumor binding peptide. Also in several embodiments of this invention the tumor targeting agent is a hormone, or a cytokine, or a growth factor, or a substance preferentially taken up by tumor cells. The art is silent on a means for treating tumors using a liposomal formulation wherein said formulation comprises a proinflammatory cytokine and one or more cancer drugs incorporated within a liposome; and wherein the exterior of said liposomes are coated with a tumor targeting agent.

### SUMMARY

This invention discloses a pharmaceutical composition for treating tumors wherein said pharmaceutical comprises a proinflammatory cytokine such as Tumor Necrosis Factor alpha (TNF-a) incorporated with one or more small molecule cancer drugs within the same liposome. The liposomes are sized to be below 250 nm in diameter to enable them to localize within the tumor due to the Enhanced Permeability and Retention (EPR) effect. This liposomal formulation will ensure the localization of the proinflammatory cytokine and the cancer drug together within the tumor and with less exposure to normal tissues. This invention also discloses that the safety and efficacy of said proinflammatory cytokine/drug liposomes could be improved by coating the exterior of said liposomes with a tumor targeting agent such as an anti-tumor antibody, or an anti-tumor aptamer, or an anti-tumor binding peptide, or a hormone, or a growth factor, or a cytokine.

### DESCRIPTION OF THE INVENTION

This invention teaches a pharmaceutical composition for treating tumors in which a proinflammatory cytokine and one or more small molecule cancer drugs are incorporated within the same liposome. It further discloses coating the exterior of said liposomes with a tumor targeting agent as a means to improve its safety and efficacy.

The following is a list of small molecule cancer drugs that are often used in chemotherapy. The list includes: altretamine, busulfan, carboplatin, carmofur, carmustine, chlorambucil, cisplatin, cyclophosphamide, dacarbazine, dactinomycin, lomustine, melphalan, oxaliplatin, temozolomide, thiotepa, 5-fluorouracil, 6-mercaptopurine, capecitabine, cytarabine, floxuridine, fludarabine, gemcitabine, hydroxyurea, methotrexate, pemetrexed, daunorubicin, doxorubicin, epirubicin, idarubicin, actinomycin-D, bleomycin, mitomycin-C, mitoxantrone, topotecan, irinotecan, etoposide, teniposide, docetaxel, estramustine, ixabepilone, paclitaxel, vinblastine, vincristine, vinorelbine. Those cancer drugs that are water-soluble can be encapsulated within the aqueous interior of the liposome while others that are lipid-soluble can be incorporated into the lipid bilayer of the liposome. It should also be noted that there are analogs of these drugs that can change their solubility profile by making lipid-soluble drugs water-soluble, and water-soluble drugs lipid-soluble. Said analogs can also be incorporated into liposomes according to their solubility profile (i.e. partition coefficient) and are therefore considered to lie within the spirit and scope of this invention.

In this invention the liposomes are prepared using one or more phospholipids selected from the following list: phosphatidylcholine (PC), egg phosphatidylcholine (EPC), hydrogenated egg phosphatidylcholine (HEPC); soy phosphatidylcholine (SPC), hydrogenated soy phosphatidylcholine (HSPC), phosphatidylethanolamine (PE), phosphatidylglycerol (PG), phosphatidylinositol (PI), monosialoganglioside and sphingomyelin (SPM); distearoylphosphatidylcholine (DSPC), dimyristoylphosphatidylcholine (DMPC), dimyristoylphosphatidylglycerol (DMPG), dipalmitoylphosphatidylcholine (DPPC), and the derivatized vesicle forming lipids such as poly(ethyleneglycol)-derivatized distearoylphosphatidylethanolamine (DSPE-PEGn where n is a polymer with a MW equal or greater than 2,000 daltons). Typically, cholesterol is included in the formulation.

In this invention the term “proinflammatory cytokine” will refer to those cytokines that are known to cause or be associated with inflammation. In particular it will include TNF-a, IL-1b, IL-6, IL-12, and IL-18. In this invention the term “tumor antigen” and/or “tumor associated antigen” will be used in the broadest sense to refer to all antigens on the tumor cell that can be targeted. In many instances the tumor antigen may not be specific to the tumor but could also be present on normal cells e.g. growth factor receptors, hormone receptors, and Cluster Determinant (CD) markers. It will also include antigens that are present in the tumor stroma. For example, extracellular material present between viable tumor cells. Another example is the growth factor receptors on vascular cells in blood vessels supplying the tumor.

The proinflammatory cytokine/drug liposome will typically have the following basic structure. A bilayer lipid membrane composed of phospholipids and cholesterol surrounding an aqueous center. As the proinflammatory cytokine is water-soluble it is encapsulated in the aqueous center of the liposome. If a water-soluble cancer drug is included in the formulation it will also be present in the aqueous center of the liposome. Alternatively, if a lipid-soluble cancer drug is included in the formulation it will be incorporated in the lipid bilayer of the liposome. In the preferred embodiment of this invention DSPE-PEGn is also included in the formulation. The DSPE portion of the DSPE-PEGn molecule is incorporated into the lipid bilayer with the PEG portion extending out into the external medium. The PEG chains extending outwards from the liposome provides steric hindrance preventing opsonins from attaching to the liposomes. This prevents the reticuloendothelial system of the patient from recognizing and removing the liposomes thus extending the bioavailability of the liposomal drug to act upon the tumor. These liposomes are typically referred to as “stealth” liposomes.

In this invention the proinflammatory cytokine/drug liposomes are sized to be between 100 nm and 250 nm in diameter. Preferably they are made to be of a uniform size of about 100-150 nm. This is to take advantage of the “Enhanced Permeability and Retention” (EPR) effect. Growing tumors are supplied by a leaky blood vasculature in which the blood capillaries have very enlarged endothelial pores that can exceed 400 nm. Liposomes that are sized to be significantly less than 400 nm can extravasate through these pores and into the tumor tissue where they will accumulate and release their contents within the tumor. The liposomes however, are too large to extravasate through the endothelial pores of normal blood capillaries and will therefore be retained within the blood circulation. This results in more of the proinflammatory cytokine and cancer drug being delivered to the tumor and less to normal tissues.

In one embodiment of this invention two or more cancer drugs are incorporated with the proinflammatory cytokine within the liposome. Incorporating multiple drugs within a liposome could result in increased cytotoxicity to the tumor without compromising safety.

This invention teaches a similar approach to preparing a number of different liposomal formulations by combining different proinflammatory cytokines with different cancer drugs. Also, that the efficacy of each liposomal composition could be further improved by coating the proinflammatory cytokine/drug combination with a tumor targeting agent.

The following examples are provide to illustrate the principles of this invention and are not to be construed as a limitation. One of ordinary skill in the art will recognize the many modifications that can be made without departing from the spirit and scope of this invention. Said changes are therefore considered to lie within the scope of this invention

### Proinflammatory Cytokine/Cancer Drug Liposomes.

### Example 1. Incorporating a Proinflammatory Cytokine and a Water-Soluble Drug within a Liposome

For purposes of illustration TNF-a is used as an example of a proinflammatory cytokine, and vincristine as an example of a water-soluble cancer drug that can be combined within a liposome. A typical example of the liposome formulation is to use a phospholipid such as hydrogenated phosphatidylcholine, cholesterol, and DSPE-PEG2000. The lipid components are dissolved in a small volume of solvent such as methanol/chloroform and placed in a rotovap to remove the solvent under vacuum and heating. The lipid residue is then hydrated using a solution of TNF-a and vincristine dissolved in distilled water or a buffer solution. The mixture is shaken and sonicated to form a coarse suspension of liposomes. This is then extruded through membranes with decreasing pore sizes using a pressure extruder to prepare liposomes of a uniform size about 100-150 nm in diameter. The process is kept at a temperature above the phase transition temperature of the lipid components of the formulation. The liposomes are then cooled to room temperature and unencapsulated TNF-a and unencapsulated vincristine are removed using column chromatography or dialysis against distilled water or buffer. The TNF-a/drug liposomes are stored in a sealed vial under an inert gas and kept in the dark at 4 C.

Other examples of water-soluble drugs that can be encapsulated within liposomes include epirubicin, idarubicin, vinblastine and vinorelbine. Also other examples of proinflammatory cytokines that can be encapsulated within liposomes include IL-1b, or IL-6, or IL-12, or IL-18. The procedure for incorporating any one of these proinflammatory cytokines with one or more water-soluble cancer drugs within a liposome is essentially the same as that described above for TNF-a and vincristine in Example 1.

Another method of encapsulating water-soluble cancer drugs within the liposome if they are amphipathic is the “pH gradient loading method”. Briefly, a pH gradient across the liposome membrane is established that will facilitate the movement of an amphipathic drug (e.g. doxorubicin) from a basic solution external to the liposome to an acidic solution within the liposome. For illustrative purposes doxorubicin is used as an example of an amphipathic drug and TNF-a as an example of the proinflammatory cytokine. The process involves two steps. First, to prepare TNF-a liposomes without the drug; and then to actively load the drug into the preformed TNF-a liposomes. Typically the liposomes are composed of hydrogenated phosphatidylcholine, cholesterol, and DSPE-PEG 2000. The lipid mixture used to prepare the liposomes are dissolved in a small volume of solvent such as methanol/chloroform and placed in a rotovap to remove the solvent under vacuum and heating. The lipid residue is hydrated using a solution of TNF-a dissolved in an acidic buffer and sonicated to form liposomes encapsulating the TNF-a. The coarse TNF-a liposome suspension is then extruded thru membranes of decreasing pore sizes using a pressure extruder until liposomes of a uniform size in diameter is achieved. Typically the liposomes will be made to be of a uniform size about 100-150 nm in diameter. The liposomes are separated from the external acidic buffer using column chromatography and the external medium is replaced with an alkaline buffer in which the amphiphatic drug such as doxorubicin is dissolved. The difference in pH between the aqueous interior of the liposome and the external medium will cause the drug to pass across the liposome membrane and concentrate within the interior of the liposome. The process is kept at a temperature that is above the phase transition temperature of the lipid components of the formulation. Unencapsulated drug is then removed using column chromatography or dialysis against distilled water or buffer. The TNF-a/drug liposomes are stored in a sealed vial under an inert gas, and kept in the dark at 4 C.

Another example of an amphiphatic drug that can be actively loaded into liposomes is irinotecan. Also other examples of proinflammatory cytokines that can be encapsulated within liposomes include IL-1b, or IL-6, or IL-12, or IL-18. The procedure for incorporating any one of these proinflammatory cytokines with irinotecan is essentially the same as that described above for preparing TNF-a/doxorubicin liposomes.

### Example 2. Incorporating a Proinflammatory Cytokine and a Lipid-Soluble Drug within a Liposome

For purposes of illustration TNF-a is used as an example of a proinflammatory cytokine and dactinomycin is used as an example of a lipid soluble drug that can be incorporated together within a liposome. Typically the liposomes are composed of hydrogenated phosphatidylcholine, cholesterol, and DSPE-PEG 2000. A lipid soluble drug such as dactinomycin is added to the lipid mixture and the drug and lipid mixture is dissolved in a small volume of organic solvent such as methanol/chloroform and placed in a rotovap to remove the solvent under vacuum and heating. The lipid residue is hydrated using a solution of TNF-a dissolved in distilled water or buffer, and shaken and sonicated to form liposomes. The coarse TNF-a/drug liposome suspension is then extruded through membranes of decreasing pore sizes using a pressure extruder to prepare liposomes of a uniform size about 100-150 nm in diameter. The process is kept at a temperature that is above the phase transition temperature of the lipid components of the formulation. The liposomes are cooled to room temperature and unencapsulated TNF-a and unencapsulated drug are removed using column chromatography or dialysis against distilled water or buffer. Note that the TNF-a is in the aqueous center of the liposome while the lipid soluble drug is incorporated in the lipid bilayer membrane of the liposome. The TNF-a/drug liposomes are stored in a sealed vial under an inert gas and kept in the dark at 4 C.

Other examples of lipid soluble cancer drugs that can be incorporated with TNF-a into liposomes include: paclitaxel, docetaxel, carmofur, etoposide and tenopside. Also other examples of proinflammatory cytokines that can be encapsulated within liposomes include IL-1b, or IL-6, or IL-12, or IL-18. The procedure for incorporating any one of these proinflammatory cytokines with one or more lipid soluble cancer drugs within a liposome is essentially the same as that described above for preparing TNF-a/dactinomycin liposomes.

In one embodiment of this invention two or more cancer drugs are combined with the pro-inflammatory cytokine within the liposome. As disclosed earlier those drugs that are water-soluble are encapsulated within the aqueous center of the liposome, and those that are lipid-soluble are incorporated into the lipid bilayer of the liposome. For purposes of illustration the following is an example of two cancer drugs—one of which is water-soluble (e.g. vincristine) and the other is lipid-soluble (e.g. dactinomycin), that are combined with a proinflammatory cytokine (e.g. TNF-a) within a liposome. The procedure for preparing these liposomes is essentially the same as that described in Example 1 with the following modification. The lipid-soluble drug dactinomycin is added to the mixture of lipids used to prepare the liposomes. The drug/lipid mixture is dissolved in methanol/chloroform solvent and dried using heat and vacuum. The drug/lipid residue is hydrated with a solution containing TNF-a and vincristine whereupon liposomes are formed in which vincristine and TNF-a are co-encapsulated within the liposome, while dactinomycin is incorporated in the lipid bilayer of the liposome. Unincorporated material is removed using column chromatography or by dialysis as described earlier and the TNF-a/multidrug liposomes are stored in a sealed vial under an inert gas and kept in the dark at 4 C.

It will be obvious to those of skill in the art that other small molecule cancer drugs can be used in lieu of vincristine and dactinomycin; and other proinflammatory cytokines can be used in lieu of TNF-a. Said modifications are therefore considered to lie within the spirit and scope of this invention.

### Tumor Targeting Proinflammatory Cytokine/Cancer Drug Liposomes.

There are basically two stages in preparing tumor targeting proinflammatory cytokine/drug liposomes. The first stage is to prepare the proinflammatory cytokine/drug liposomes; and the second stage is to attach a tumor targeting agent to the exterior of said liposomes. There are two methods whereby the tumor targeting agent is attached. One method is to incorporate a linking molecule such as DSPE-PEG-maleimide (DSPE-PEG-MAL) into the liposome formulation such that when the liposomes are formed the DSPE portion of the molecule is embedded within the bilayer of the liposome with the maleimide site exposed to the external medium. The tumor targeting agent is then attached to the active site on the maleimide thus anchoring it to the liposome. In this invention this procedure will be termed the “direct method” of attachment. The other method is to prepare the proinflammatory cytokine/drug liposomes without including the DSPE-PEG-MAL in the formulation. The tumor targeting agent is attached to the DSPE-PEG-MAL in a separate reaction. The DSPE-PEG-MAL-tumor targeting agent complex is then incubated with the proinflammatory cytokine/drug liposomes at a temperature above the phase transition temperature of the liposomal lipids, whereupon the DSPE portion of the complex will embed within the lipid bilayer of the liposome thus anchoring the complex to the liposome. The tumor targeting agent is thereby attached to the liposome. In this invention this procedure will be termed the “post-insertion” method of attachment.

The following examples are provided for illustration and are not to be construed as a limitation. One of ordinary skill in the art will recognize many modifications that can be made without departing form the spirit and scope of this invention. Said changes are therefore considered to lie within the scope of this invention.

### Example 3. Tumor Targeting Proinflammatory Cytokine/Drug Liposomes Prepared Using the “Direct Method” of Attachment

For purposes of illustration TNF-a is selected as the proinflammatory cytokine, vincristine as the cancer drug, and anti-Epidermal Growth Factor Receptor (EGFR) antibody as the targeting agent. The TNF-a/drug liposomes are prepared as described earlier in Example 1 with the following modification to the original formulations. A small amount of DSPE-PEG-MAL is added to the mixture of phospholipids, cholesterol and DSPE-PEG2000 used to prepare the liposomes. The liposomes thus prepared with have the DSPE portion of the DSPE-PEG-MAL embedded in the bilayer membrane with the MAL portion exposed to the external medium and available for attachment to the tumor targeting agent.

To prepare the tumor targeting antibody in a form suitable for attachment to the maleimide site it is first fragmented into the Fab and Fc fragments using immobilized papain. The Fc fragment is then removed using immobilized Protein A leaving purified Fab in solution. The Fab is then incubated with the TNF-a/drug liposomes where it will bind to the exposed maleimide site on the DSPE-PEG-MAL molecule and thus become attached to the liposome. Any unattached Fab is removed using column chromatography or dialysis against distilled water or buffer. The tumor targeting TNF-a/drug liposomes are stored in a sealed vial under an inert gas and kept in the dark at 4 C.

It will be obvious to those of skill in the art that other small molecule cancer drugs can be used in lieu of vincristine, and other proinflammatory cytokines such as IL-1b, or IL-6, or IL-12, or IL-18 can be used in lieu of TNF-a; and that each of these proinflammatory cytokine/drug liposomal combinations can be coated with a tumor targeting agent using the “direct” method of attachment. Also that liposomes incorporating a cytokine (e.g. TNF-a) and multiple drugs can similarly be coated with a targeting agent using the “direct” method. Said proinflammatory cytokine/drug permutations coated with a targeting agent are considered to lie within the spirit and scope of this invention.

### Example 4. Tumor Targeting TNF/Drug Liposomes Prepared Using the “Post-Insertion Method” of Attachment

For purposes of illustration TNF-a is selected as the proinflammatory cytokine, vincristine as the cancer drug, and anti-Epidermal Growth Factor Receptor (EGFR) antibody as the targeting agent. The TNF-a/drug liposomes are prepared as described earlier in Example 1. To prepare the tumor targeting antibody in a form suitable for attachment to the maleimide site it is first fragmented into the Fab and Fc fragments using immobilized papain. The Fc fragment is then removed using immobilized Protein A leaving purified Fab in solution. The Fab is then incubated with DSPE-PEG-MAL and will bind to the MAL site to form a DSPE-PEG-MAL-Fab complex. The DSPE-PEG-MAL-Fab complex is then incubated with the preformed liposomes at an elevated temperature (e.g. 65 C for 30 minutes) to allow the DSPE portion of the complex to embed within the lipid bilayer of the liposome with the Fab portion exposed to the external medium. Any unattached DSPE-PEG-MAL-Fab is removed using column chromatography or dialysis against distilled water or buffer. The tumor targeting TNF-a/drug liposomes are stored in a sealed vial under an inert gas and kept in the dark at 4 C.

It will be obvious to those of skill in the art that other small molecule cancer drugs can be used in lieu of vincristine, and other proinflammatory cytokines such as IL-1b, or IL-6, or IL-12, or IL-18 can be used in lieu of TNF-a; and that said liposomes can be coated with a targeting agent using the “post-insertion” method without departing from the spirit and scope of this invention. Also that liposomes incorporating a cytokine (e.g. TNF-a) and multiple drugs can similarly be coated with a targeting agent using the “post-insertion” method. Said proinflammatory cytokine/drug permutations coated with a targeting agent are considered to lie within the spirit and scope of this invention.

### Tumor Targeting Agents

Many of the tumor targeting agents used today are tumor targeting antibodies. However, there are a variety of other types of tumor targeting agents that can be used to target tumors. For example aptamers and binding peptides have binding capabilities that mimic the action of antibodies. There are also ligands such as hormones and cytokines that can target cellular receptors on tumor cells; and there are substances such as folic acid and transferrin that are preferentially taken up by tumors. In this invention the term “tumor targeting agents” will be used in the broadest sense to include all substances capable of binding to the tumor, or to the tumor stroma e.g. tumor vasculature. These will include anti-tumor antibodies, anti-tumor aptamers and anti-tumor binding peptides. It will also include hormones, cytokines, growth factors and substances preferentially taken up by tumors.

### Antibody:

In this invention the term “antibody” will include polyclonal, monoclonal and recombinant antibodies, and the binding site fragments of those antibodies. Polyclonal antibodies are produced by immunizing animals such as rabbits, goats and horses with a tumor associated antigen and collecting the antiserum. The antiserum is processed using established methods such as salt-fractionation, gel chromatography and affinity purification to prepare a purified anti-tumor antibody. Monoclonal antibodies are prepared using hybridoma technology using mice, rabbit, human or other cell lines. When prepared in other species they are often “humanized” by replacing certain components of the monoclonal antibody molecule with human components. Recombinant antibodies are produced using genetic engineering techniques in which the genetic code for the antibody is identified and then expressed in genetically modified bacteria, or fungi, or insect and mammalian cells lines. These and other methods of producing purified anti-tumor antibodies are well-known to those of skill in the art and are considered to lie within the scope of this invention. In this invention the term antibody refers to the whole antibody molecule, and/or the binding fragments Fab and F(ab)2; and/or to recombinant single chain binding fragments (scFv).

In one embodiment of this invention the tumor targeting antibody is an antibody that targets Human Epidermal Growth Factor 2 Receptors (HER2) that are over-expressed in some breast cancers. For example, Herceptin® (trastuzumab) is a commercially available humanized monoclonal antibody that targets HER2 and there are biosimilar versions being developed. Anti-HER2 antibody and biosimilar versions can be used to prepare a tumor targeting liposomal proinflammatory cytokine/drug formulation using the general methods described in this invention. Tumor targeting liposomes prepared using anti-HER2 antibody will have the capacity to bind to breast cancer cells and anchor the liposomes within the tumor where the proinflammatory cytokine and drug are released for maximum effect.

In one embodiment of this invention the tumor targeting antibody is an antibody that targets Human Epidermal Growth Factor Receptors (EGFR) present on cancer cells. For example, Erbitux® (cetuximab) is a commercially available chimeric human/mouse monoclonal antibody that will target EGFR over-expressed in colorectal cancer and squamous cell carcinoma of the head and neck. Vectibix® (panitumumab) is a fully human monoclonal antibody that also targets EGFR in metastatic colorectal cancer. Anti-EGFR antibody and biosimilar versions can be used to prepare a tumor targeting liposomal proinflammatory cytokine/drug formulation using the general methods described in this invention. Tumor targeting liposomes prepared using anti-EGFR antibody will have the capacity to bind to the cancer cells and anchor the tumor targeting liposomes within the tumor where the proinflammatory cytokine and drug are released for maximum effect.

In one embodiment of this invention the tumor targeting antibody is not directed to a tumor antigen but instead it targets the proliferating vascular cells in the blood vessels supplying the tumor. The antibody targets Vascular Endothelial Growth Factor Receptors (VEGFR) present on proliferating vascular cells. The Fab fragment of the anti-VEGFR antibody is prepared and attached to the proinflammatory cytokine/drug liposomes as described earlier. When injected into the cancer patient the VEGFR targeting proinflammatory cytokine/drug liposomes will bind to and kill the proliferating vascular cells in the blood vessels supplying the tumor. The blood supply to the tumor is interrupted and tumor growth is inhibited.

In one embodiment of this invention the tumor targeting antibody is an autoimmune antinuclear antibody (ANA) that targets the extracellular nuclear material present in the necrotic regions of solid tumors. The ANA is collected from patients with systemic lupus erythematosus (SLE) and purified using salt-fractionation and immunoaffinity methods. The Fab fragment of the antibody is prepared and attached to the proinflammatory cytokine/drug liposome thru a DSPE-PEG-MAL moiety as described earlier. When tumor targeting ANA liposomes are injected into the cancer patient the antinuclear antibody will bind to extracellular nuclear antigens present in the necrotic areas of the tumor and thus anchor the ANA liposomes within the tumor where the proinflammatory cytokine and drug are released for maximum effect. As almost all solid tumors will have areas of necrosis the ANA proinflammatory cytokine/drug liposomes may be utilized to treat a wide variety of different types of solid tumors.

There are a growing number of new anti-tumor antibodies being developed that can be used to prepare tumor targeting liposomes. The tumor associated antigens that can be targeted include a variety of hormone receptors, growth factor receptors, cytokine receptors, and cell-surface markers such as Cluster Determinants (CD) present on tumor cells. These antibodies can be prepared and attached to proinflammatory cytokine/drug liposomes using the general principles outlined in this invention.

It will be obvious to those of skill in the art that in addition to tumor targeting antibodies there are a wide variety of other types of targeting agents such as aptamers and binding peptides that can be used in lieu of antibodies to target the tumor.

### Aptamer:

Aptamers are small (i.e. 40-100 bases), synthetic single-stranded oligonucleotides (ssDNA or ssRNA) that can specifically recognize and bind to virtually any kind of target, including ions, whole cells, drugs, toxins, low-molecular-weight ligands, peptides, and proteins. Each aptamer has a unique configuration as a result of the composition of the nucleotide bases in the chain causing the molecule to fold in a particular manner. Because of their folded structure each aptamer will bind selectively to a particular ligand in a manner analogous to an antibody binding to its antigen. Aptamers are usually synthesized from combinatorial oligonucleotide libraries using in vitro selection methods such as the Systematic Evolution of Ligands by Exponential Enrichment (SELEX). This is a technique used for isolating functional synthetic nucleic acids by the in vitro screening of large, random libraries of oligonucleotides using an iterative process of adsorption, recovery, and amplification of the oligonucleotide sequences. The iterative process is carried out under increasingly stringent conditions to achieve an aptamer of high affinity for a particular target ligand. In order to improve stability against nucleases found in vivo the oligonucleotides may be modified to avoid nuclease attack. They may for example be synthesized as L-nucleotides instead of the natural D-nucleotides and thus avoid degradation from the natural nucleases. The aptamer can be synthesized with a thiol (S—S)-modified 5′ end to enable it to bind to the maleimide site of the DSPE-PEG-MAL polymer and thus become attached to surface of the proinflammatory cytokine/drug liposome.

In one embodiment of this invention the tumor targeting agent is an anti-human epidermal growth factor receptor 2 (HER 2) aptamer.

In one embodiment of this invention the tumor targeting agent is an anti-epidermal growth factor receptor (EGFR) aptamer.

In one embodiment of this invention the tumor targeting agent is an anti-vascular endothelial growth factor receptor (VEGFR) aptamer.

In one embodiment of this invention the tumor targeting agent is an anti-nuclear aptamer.

### Binding Peptide:

Binding peptides consist of a chain of aminoacids that fold in such a manner that their configuration makes them capable of binding to antigens in a manner that mimics the binding of an antibody to its antigen. There are various well-known methods for preparing synthetic or biological peptide libraries composed of up to a billion different sequences, and for identifying a particular peptide sequence that will target a particular antigen. The binding peptide can be produced with a thiol group at one end to enable it to bind to the maleimide site of the DSPE-PEG-MAL polymer and thus become attached to the surface of the proinflammatory cytokine/drug liposome.

In one embodiment of this invention the tumor targeting agent is an anti-human epidermal growth factor receptor 2 (HER 2) binding peptide.

In one embodiment of this invention the tumor targeting agent is an anti-epidermal growth factor receptor (EGFR) binding peptide.

In one embodiment of this invention the tumor targeting agent is an anti-vascular endothelial growth factor receptor (VEGFR) binding peptide.

In one embodiment of this invention the tumor targeting agent is an anti-nuclear binding peptide.

Other examples of targeting agents include ligands such as hormones, cytokines and growth factors. Cells communicate by producing biological messengers such as hormones, cytokines and growth factors that bind to their specific receptors on cells causing them to respond in some fashion. These ligands can be utilized to prepare tumor targeting liposomes that will target tumor cells bearing specific receptors. For example, a hormone such as estrogen attached to the liposome can be used to target estrogen receptive breast cancer cells. Similarly a hormone such as progesterone attached to the liposome can be used to target progesterone receptive breast cancer cells. Similarly a cytokine such as VEGF attached to the liposome can be used to target VEGF receptors present on proliferating vascular cells in blood vessels supplying the tumor. The means of attaching these ligands to the liposome are well-known to those of skill in the art.

In one embodiment of this invention the tumor targeting agent is estrogen.

In one embodiment of this invention the tumor targeting agent is progesterone.

In one embodiment of this invention the tumor targeting agent is Vascular Endothelial Growth Factor (VEGF).

Finally there are examples of certain substances such as folic acid and transferrin that appear to be selectively taken up by cancer cells compared to normal cells. These can be utilized as tumor targeting agents by attaching them to the proinflammatory cytokine/drug liposomes. The means of attaching these compounds to the liposome are well-known to those of skill in the art.

In one embodiment of this invention the tumor targeting agent is folic acid.

In one embodiment of this invention the tumor targeting agent is transferrin.

This invention teaches a means of treating tumors using a pharmaceutical composition in which a proinflammatory cytokine and one or more small molecule cancer drugs are both incorporated within a liposome. This combination will have a synergistic cytotoxic effect upon the tumor with less harm to normal tissues. This invention also teaches that attaching a tumor targeting agent to said liposomes will further improve the safety and efficacy of said liposomes. One of skill in the art would be aware from the teachings in this invention that there are many modifications that can be made without departing from the spirit and scope of this invention. Said modifications and changes made as a result of the teachings in this invention are therefore considered to lie within the scope of this invention.

### REFERENCES

Brown T D, Goodman P, Fleming T, Macdonald J S, Hersh E M, Braun T J. A phase II trial of recombinant tumor necrosis factor in patients with adenocarcinoma of the pancreas: a Southwest Oncology Group study (1991). J Immunother. 10:376-8.
Carswell, E. A., Old, L. J., Kassel, R. L., Green, S., Fiore, N., and Williamson, B. (1975) An endotoxin-induced serum factor that causes necrosis of tumors. Proc Natl Acad Sci USA 72, 3666-3670
Creagan E T, Kovach J S, Moertel C G, Frytak S, and Kvols L K, (1988) A, phase I clinical trial of recombinant human tumor necrosis factor. Cancer 62, 2467-71
Creasey A A, Reynolds M T, and Laird W, (1986) Cures and partial regression of murine and human tumors by recombinant human tumor necrosis factor. Cancer Res 46, 5687-90
Curnis F, Sacchi A, and Corti A, (2002) Improving chemotherapeutic drug penetration in tumors by vascular targeting and barrier alteration. J Clin Invest 110, 475-82
de Wilt J H, Manusama E R, van Tiel S T, van Ijken M G, ten Hagen T L, and Eggermont A M, (1999) Prerequisites for effective isolated limb perfusion using tumour necrosis factor α and melphalan in rats. Br J Cancer 80, 161-6
Eggermont A M, (1996) The success of TNF α in isolated limb perfusion for irresectable extremity soft tissue sarcomas melanoma and carcinomas: observations in patients and preclinical perfusion models. Gan To Kagaku Ryoho 23 1357-70.
Eggermont A M, et al. (1996) Isolated limb perfusion with high-dose tumor necrosis factor-alpha in combination with interferon-gamma and melphalan for nonresectable extremity soft tissue sarcomas: a multicenter trial. J Clin Oncol. 4:2653-2665
Fraker D L, Alexander H R, Andrich M, Rosenberg S A. (1996). Treatment of patients with melanoma of the extremity using hyperthermic isolated limb perfusion with melphalan, tumor necrosis factor, and interferon gamma: results of a tumor necrosis factor dose-escalation study. J Clin Oncol. 14:479-489.
Furman W L, Strother D, McClain K, Bell B, Leventhal B, and Pratt C B, (1993) Phase I clinical trial of recombinant human tumor necrosis factor in children with refractory solid tumors: A, Pediatric Oncology Group study. J Clin Oncol 11, 2205-10.
Lejeune F J. (1995) High dose recombinant tumour necrosis factor (rTNF alpha) administered by isolation perfusion for advanced tumors of the limbs: a model for biochemotherapy of cancer. Eur J Cancer. 31A:1009-1016.
Lienard D, Ewalenko P, Delmotte J J, Renard N, Lejeune F J. (1992) High-dose recombinant tumor necrosis factor alpha in combination with interferon gamma and melphalan in isolation perfusion of the limbs for melanoma and sarcoma. J Clin Oncol. 0:52-60.
Maruyama K. Intracellular targeting delivery of liposomal drugs to solid tumors based on EPR effects. (2011) Adv Drug Deliv Rev. March 18; 63(3):161-9.
Selby P, Hobbs S, Viner C, Jackson E, Jones A, Newell D, Calvert A H, McElwain T, Fearon K, Humphreys J, and et al (1987) Tumour necrosis factor in man: clinical and biological observations. Br J Cancer 56, 803-8.
Watanabe N, Niitsu Y, Umeno H, Kuriyama H, Neda H, Yamauchi N, Maeda M, and Urushizaki I (1988) Synergistic cytotoxicity of recombinant human TNF and various anti-cancer drugs. Immunopharmacol Immunotoxicol 10, 117-27

What is claimed is:

1. A method for treating tumors by administering to the cancer patient in need a therapeutic liposomal biopharmaceutical comprising a proinflammatory cytokine and one or more small molecule cancer drugs incorporated together within a liposome; and wherein the exterior of said liposome is coated with a tumor targeting agent.

2. A method for treating tumors according to claim 1 wherein the proinflammatory cytokine is tumor necrosis factor alpha (TNF-a).

3. A method for treating tumors according to claim 1 wherein the one or more small molecule cancer drugs are selected from the following list: altretamine, busulfan, carboplatin, carmofur, carmustine, chlorambucil, cisplatin, cyclophosphamide, dacarbazine, dactinomycin, lomustine, melphalan, oxaliplatin, temozolomide, thiotepa, 5-fluorouracil, 6-mercaptopurine, capecitabine, cytarabine, floxuridine, doxorubicin, fludarabine, gemcitabine, hydroxyurea, methotrexate, pemetrexed, daunorubicin, epirubicin, idarubicin, actinomycin-D, bleomycin, mitomycin-C, mitoxantrone, topotecan, irinotecan, etoposide, teniposide, docetaxel, estramustine, ixabepilone, paclitaxel, vinblastine, vincristine, vinorelbine.

4. A method for treating tumors according to claim 1 wherein said liposome is composed of a) one or more phospholipids selected from the following list: phosphatidylcholine (PC), egg phosphatidylcholine (EPC), hydrogenated egg phosphatidylcholine (HEPC); soy phosphatidylcholine (SPC), hydrogenated soy phosphatidylcholine (HSPC), phosphatidylethanolamine (PE), phosphatidylglycerol (PG), phosphatidylinositol (PI), monosialoganglioside and sphingomyelin (SPM); distearoylphosphatidylcholine (DSPC), dimyristoylphosphatidylcholine (DMPC), dimyristoylphosphatidylglycerol (DMPG), dipalmitoylphosphatidylcholine (DPPC), and the derivatized vesicle forming lipids such as poly(ethyleneglycol)-derivatized distearoylphosphatidylethanolamine (DSPE-PEGn where n is a polymer with a MW equal or greater than 2,000 daltons), and b) cholesterol.

5. A method for treating tumors according to claim 1 wherein the tumor targeting agent is an anti-epidermal growth factor receptor (EGFR) antibody, or an anti-epidermal growth factor receptor (EGFR) aptamer, or an anti-epidermal growth factor receptor (EGFR) binding peptide.

6. A method for treating tumors according to claim 1 wherein the tumor targeting agent is an anti-human epidermal growth factor receptor 2 (HER2) antibody, or an anti-human epidermal growth factor receptor 2 (HER2) aptamer, or an anti-human epidermal growth factor receptor 2 (HER2) binding peptide.

7. A method for treating tumors according to claim 1 wherein the tumor targeting agent is an anti-nuclear antibody (ANA), or an anti-nuclear aptamer, or an anti-nuclear binding peptide.

8. A method for treating tumors according to claim 1 wherein the tumor targeting agent is an anti-vascular endothelial growth factor receptor (VEGFR) antibody, or an anti-vascular endothelial growth factor receptor (VEGFR) aptamer, or an anti-vascular endothelial growth factor receptor (VEGFR) binding peptide.

9. A method for treating tumors according to claim 1 wherein the tumor targeting agent is estrogen.

10. A method for treating tumors according to claim 1 wherein the tumor targeting agent is progesterone.

11. A method for treating tumors according to claim 1 wherein the tumor targeting agent is vascular endothelial growth factor (VEGF).

12. A method for treating tumors according to claim 1 wherein the tumor targeting agent is folic acid.

13. A method for treating tumors according to claim 1 wherein the tumor targeting agent is transferrin.

14. A method for treating tumors according to claim 1 wherein a therapeutic dosage of said liposomal biopharmaceutical is administered intravenously by injection or by infusion into a cancer patient in need.
